(19) United States
(12) Patent Application Publication
(10) Pub. No.: US 2007/0156036 A1
(43) Pub. Date: Jul. 5, 2007

(54) TIME-RESOLVED NON-INVASIVE OPTOMETRIC DEVICE FOR DETECTING DIABETES

(75) Inventors: Laurent G. Pilon, Los Angeles, CA (US); Kamal M. Katika, Los Angeles, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY OF CALIFORNA, Oakland, CA (US)

(21) Appl. No.: 11/610,423

(22) Filed: Dec. 13, 2006

Related U.S. Application Data
(63) Continuation of application No. PCT/US05/21588, filed on Jun. 17, 2005.
(60) Provisional application No. 60/581,123, filed on Jun. 17, 2004.

(51) Int. Cl. A61B 5/00

(52) U.S. Cl. 600/310; 600/317

(57) ABSTRACT

A time-resolved fluorescence device is described for the detection and diagnosis of diabetes in a noninvasive manner. The device uses an ultra-short excitation pulse of light in the UV, infrared or visible range that comprises of a repetition of nanosecond pulses. The excitation pulse is directed incident onto a strategically selected area of the patient body such as the forearm, the feet, and the palm. This light interacts with the different layers of the skin. The absorbed light excites the AGEs in the skin, which in turn generate a fluorescence signal, which is collected by a detector. A processor is coupled to the detector to measure the transient fluorescence intensity decay of the skin in terms of lifetimes, and the contribution of individual fluorophores to the overall fluorescence signal. The nature and location of the fluorophores may be identified and a medical diagnostics may be performed.

### CROSS-REFERENCE TO RELATED APPLICATIONS

This application claims priority from, and is a 35 U.S.C. § 111(a) continuation of, co-pending PCT international application serial number PCT/US2005/021588, filed on Jun. 17, 2005, incorporated herein by reference in its entirety, which claims priority from U.S. provisional application Ser. No. 60/581,123, filed on Jun. 17, 2004, herein incorporated by reference in its entirety.

This application is related to PCT International Publication Number WO/2006/009910 A2, herein incorporated by reference in its entirety, and to PCT International Publication Number WO/2006/009906 A2, herein incorporated by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to a non-invasive diabetes diagnostic and detection, and more particularly to time-resolved optometric measurements for diagnostic and detection of diabetes.

2. Description of Related Art

There are three main kinds of diabetes. Type 1 diabetes, or insulin-dependent diabetes, is usually first diagnosed in children, teenagers, or young adults. In this form of diabetes, the beta cells of the pancreas no longer make insulin because the body's immune system has attacked and destroyed them.

Type 2 diabetes, also known as non-insulin-dependent diabetes, is the most common form of diabetes. People can develop type 2 diabetes at any age. This form of diabetes usually begins with insulin resistance, a condition in which fat, muscle, and liver cells do not use insulin properly because they are no longer sensitive to it. At first, the pancreas keeps up with the added demand by producing more insulin. In time, however, it loses the ability to secrete enough insulin in response to meals.

Finally, women may develop gestational diabetes during the late stages of pregnancy. Although this form of diabetes usually goes away after the baby is born, a woman who has had it is more likely to develop type 2 diabetes later in life. Gestational diabetes is caused by the hormones of pregnancy or a shortage of insulin.

In addition to these three traditional types of diabetes, there is an emergence of maturity onset diabetes of youth (MODY). The various types of MODY are due to mutations in specific transcription factors important in the pancreas, are inherited in an autosomal dominant manner, and are seen increasingly in obese teenagers.

The number of people with diabetes worldwide has tripled since 1985 to reach 194 million in 2003. By 2025, the number of people with diabetes is expected to more than double in Africa, the Eastern Mediterranean and Middle East, and South-East Asia, and rise by 20% in Europe, 50% in North America, 85% in South and Central America and 75% in the Western Pacific.

In the United States, more than 18 million people were afflicted with diabetes mellitus in 2002. One third of which (i.e., 6 million) remain undiagnosed. The number of diabetic population continues increasing, and an estimated 23 million Americans will have diabetes by 2010. Diabetes is particularly common in ageing populations, thus affecting countries around the globe whose population tends to live longer. In addition, children are developing Type 2 diabetes in developed countries, once thought to only occur in adults.

Moreover, some 314 million people, or 8.2% in the global adult population, are estimated to have impaired glucose tolerance (IGT), a state which often precedes diabetes. In many cases, the patient's blood glucose levels are higher than normal, but not high enough for a diagnosis of diabetes.

The medical complications associated with diabetes are quite serious. Diabetes is the leading cause of blindness, kidney failure, macrovascular disease, and lower limb amputation. Complications of diabetes claim the life of about 200,000 Americans every year. Type 2 diabetes results in premature death reducing the patient's lifetime by about 15 years

Diabetes can be considered a worldwide epidemic whose financial cost is tremendous and steadily increasing. The cost of diabetes on the US health care system alone was estimated at more than $132 Billions in 2002 due to medical expenditures and lost of. Early detection of diabetic patients would not only reduce its human cost by limiting the extent of irreversible effect of diabetes, but also its economic costs.

Current screening tests for diabetes consist of Fasting Plasma Glucose (FGP) and Oral Glucose Tolerance (OGT). The FGP test is performed after a person has fasted for at least 8 hours. Fasting stimulates the release of the hormone glucagon, which in turn raises plasma glucose levels. In people without diabetes, the body will produce and process insulin to counteract the rise in glucose levels. In people with diabetes, this does not happen, and the tested glucose levels will remain high. Typically, a sample of blood is taken from a vein in the arm. If the blood glucose level is greater than or equal to 126 mg/dl, the person is retested and, if the results are consistent, diagnosed with diabetes. Individuals with a fasting plasma glucose level less than 126 mg/dl, but greater than or equal to 110 mg/dl, are classified as having impaired fasting glucose. Though they do not have diabetes, these individuals do not metabolize glucose normally, and they have an increased risk of developing high blood pressure, blood lipid disorders, and Type 2 diabetes.

The OGT test is performed after an overnight fast, and the patient drinks a solution containing a known amount of glucose. Blood is obtained before the patient drinks the glucose solution, and blood is drawn again every 30 to 60 minutes after the glucose is consumed for up to 3 hours.

The currently available tests present the following disadvantages: 1) they require the patient to fast overnight; 2) they require a long period of time in which the patient has to remain seated (which maybe difficult for young and elderly patients); 3) they are generally invasive measurements in the forearm that draw blood, causing patient discomfort; 4) they are not practical for routine, random testing, or pre-screening (early detection).

Hyperglycemia found in patients with type 2 diabetes mellitus alters the structure of long-lived proteins, including the two main structural proteins in the skin: elastin and collagen. These proteins are damaged by the formation of Nonenzymatic glycosylation (NEG) of proteins associated with hyperglycemia. NEG (or glycation) is a nonenzymatic post-translational modification of proteins, resulting from chemical reactions between glucose and the primary amino groups of the proteins. Glucose initially reacts with proteins in a reversible manner to create early glycation products such as fructoselysine (FL) and other Amadori products. This is the first step in a series of reactions collectively called the Maillard reaction. The latter is responsible for the formation of Advanced Glycation End (AGE) products.

Advanced Glycation End products (AGE) products accumulate in tissues including arterial walls, skin, tendons, lung, and the lens capsule basement membrane and alter their properties. AGE products also accumulate in long lived proteins, such as vascular collagen, and reduce the elasticity (i.e., increase stiffness) of vessel walls. Thus, diabetes also has an effect on the skin blood vessels that becomes atrophied.

One important characteristic of AGEs in terms of detection is that they cause the skin of inadequately controlled diabetic patients to fluoresce significantly more than that of treated patients and healthy subjects of the same age. It has been established, both in-vitro and in-vivo, that the intensity of the fluorescent signal from the level of skin AGEs highly correlates with the duration and severity of hyperglycemia and with the presence of long term diabetic complications as well as with aging (e.g., Brownlee M., Cerami A. and Vlassara H., 1988. Advanced glycosylation end products in tissue and the biochemical basis of complications of diabetes. New England Journal of Medicine, Vol. 318, pp.1315-1321).

Thus, an autofluorescence “signature” of AGE accumulated in the skin may be obtained that reflects the quality of long term glycemic control, and of the patient's risks of developing diabetes and its complications. The further quantification of the presence and concentration of skin AGEs may also provide a measure of hyperglycemia over several years.

Studies on model compounds in vitro have demonstrated that the excitation/emission maxima of various AGEs do not differ considerably from one another. All compounds studied have the excitation maximum between 335 nm and 370 nm and the emission maximum between 385 nm and 440 nm which makes multicomponent analysis by spectrofluorometry difficult (Deyl Z., I. Mik\check{s}ík, J. Zicha and D. Jelínková, 1997. Reversed-phase chromatography of pentosidine-containing CNBr peptides from collagen, Analytica Chimica Acta, Vol. 352, pp. 257-270).

Very recently, a steady-state autofluorescence reading device was developed for assessing the accumulation of advanced glycation end products in skin (Meerwaldt, R., R. Graaff, P. H. N. Oomen, T. P. Links, J. J. Jager, N. L. Alderson, S. R. Thorpe, J. W. Baynes, R. O. B. Gans, A. J. Smith, 2004. Simple non-invasive assessment of advanced glycation endproduct, accumulation, Diabetologia, Vol. 47, pp. 1324-1330). The wavelength of the excitation source was varied between 300 nm and 420 nm and the fluorescent signal was measured between 300 nm and 600 nm. The fluorescence signal was found to correlate with the presence of several key AGEs in the skin, as well as with diabetes duration, mean HbA1C of the previous year, and creatinine levels. However, the vast majority of the human subjects were Caucasian, and measurements were performed only on the patient's forearm. Moreover, steady-state fluorescence techniques of the above device have several disadvantages that limit their effectiveness: 1) they cannot distinguish fluorophores emitting at similar wavelengths; 2) they are influenced by endogeneous chromophores, which interact with the excitation and fluorescent light; and 3) the fluorescence signal depends on the geometry and the probe design, and the properties of the skin such as pigmentation.

Accordingly, an object of the present invention is to provide a time-resolved photometric device and the associated analysis software for early detection of diabetes in a non-invasive, reliable, cheap, and convenient manner.

A further object is to provide means for assessing long term blood glucose control in patients with diabetes to prevent abnormal AGE accumulation.

Another object is to provide means to monitor the efficacy of therapy and provide insight into the causes and treatment of diabetic complications.

At least some of the above objects will be met in the invention described hereafter.

### BRIEF SUMMARY OF THE INVENTION

A time-resolved fluorescence device is described for the detection and diagnosis of diabetes in a noninvasive manner. The device can also be used for monitoring the efficacy of therapy and provides insight into the causes and treatment of diabetic complications. The device uses an excitation pulse of electromagnetic (EM) wave (such as UV, IR or visible light) that comprises of a repetition of pulses (time resolution) as opposed to shining the excitation light on the patient's skin continuously (steady state). The pulse width is selected in such a way that it is much smaller than the fluorescence lifetime of the molecules or protein of interest. The excitation pulse is directed incident onto a strategically selected area of the patient body such as the forearm, the feet, and the palm. The pulse of excitation light is partially absorbed and scattered by the different skin layers. The absorbed light excites some proteins and the AGEs in the skin which in turn generate a fluorescence signal, which is collected by a receiving detector, converted to an electrical signal, and then analyzed. A processing unit analyzes the transient fluorescence signal of the skin in terms of lifetimes, quantum yields, and/or the fraction of individual fluorophores contribution to the overall or specific variables of the fluorescence signal, as well as their absolute or relative local concentrations in the skin.

The device can also monitor simultaneously the reflected and transmitted light intensity as a complementary and alternative approach. The temporal signals are then preferably processed using an inverse method developed based on transient propagation of light in multilayer biological tissues. The signal generated by the methods of the present invention is strong enough and sensitive enough to detect and differentiate the fluorescence emission from proteins in the skin including that of AGEs resulting from the Maillard reactions due to tissues' exposure to glucose.

Time resolved fluorescence techniques include, but are not limited to, Time-Correlated Single Photon Counting (TCSPC), frequency modulation, gated photon counter, or the like. Design parameters include, but are not limited to, the energy, excitation pulse width, wavelengths of the excitation light and of the detection as well as repetition rate, detector settings, modulation frequency, gate width, etc. The areas of the body ideally suited to be probed include, but are not limited to, the forearms, the palms, the feet, the earlobes, and the skin flap between the thumb and the forefinger. The method of the present invention enables the determination of the type, location, and relative concentration of the fluorophores. Based on the above data, medical diagnostic may then be performed. The device and software of the present invention are small and portable allowing for earlier and regular prescreening for diabetes. In addition, it can also be applied to other diseases affecting the optical properties of skin.

The time-resolved system of the present invention eliminates many of the limitations of currently available (steady-state) systems. In particular, because different fluorophores have different lifetimes, they can be identified and their location in the skin can be determined by processing the temporal signals. Finally, the time-resolved measurements are not as sensitive to the variations in the condition of the skin (e.g., motion artifacts, pigmentation, hair, and suntan) as the steady-state method.

In one aspect of the invention, a method is disclosed for non-invasively detecting diabetes in a patient. The method includes the steps of directing an excitation pulse of light at a region of the patient's skin, and exciting one or more AGE products in the skin, wherein excitation of said one or more AGE products generates a fluorescence signal. The method further includes detecting the fluorescence signal generated by the one or more AGE products, and measuring the fluorescence signal as a function of time.

In one embodiment, a plurality excitation pulses (such as UV or IR light) are repeatedly directed in succession at the region of the patient's skin. Typically, the excitation pulses are subjected on the patient's skin at a rate of at least 1 MHz. Preferably, the pulses are directed at a rate of at least 5 MHz.

In another embodiment, the reflectance and transmittance of the excitation pulse of light may be measured at the sensing region. Furthermore, the transmittance, reflectance, and time-resolved fluorescence measurements may be performed simultaneously.

In another embodiment of the current aspect, the method includes storing fluorescence signal values acquired from a plurality of reference patients in a database. Then the measured fluorescence signal may be compared to the stored fluorescence signal (e.g. intensity decay) values indicative of diabetes. The compared fluorescence signal may also be used to assess the long term glycemic control in the patient, or to assess the impaired glucose tolerance in the patient.

In another embodiment, one or more fluorophores may be identified from the measured in-vivo fluorescence signal.

Another aspect is an apparatus for detecting diabetes in a patient. The apparatus has an excitation source configured to direct electromagnetic excitation energy at a region of the patient's skin, and a detector directed at the region of skin. The detector is configured to receive a fluorescence signal resulting from the excitation energy at the patient's skin. The apparatus further includes a processor configured to measure intensity decay of the fluorescence signal as a function of time to diagnose the diabetic condition of the patient.

In a preferred embodiment, excitation source comprises one or more LEDs.

Another aspect of the invention is a method for performing time-resolved fluorescence measurements to diagnose the diabetic condition of a patient. The method comprises: directing an excitation pulse at a region of the patient's skin; exciting a portion of the patient's skin as a result of the excitation pulse at the region to generate a fluorescence signal indicative of the composition of the patient's skin; detecting the fluorescence signal generated by the excitation pulse; and measuring a transient intensity decay of the fluorescence signal to determine the diabetic condition of the patient.

In one embodiment, exciting one or more AGE products are excited in the skin, the AGE products each generating a fluorescence signal.

In many embodiments, a plurality of ultra short pulses may be directed in succession at the region of the patient's skin, or a frequency modulated light may be repeatedly directed at the region of the patient's skin. The signals from the successive pulses may be added to increase the signal-to noise ratio of the signal.

In another embodiment, the method may further include distinguishing between the one or more AGE products by measuring their emission wavelengths. Distinguishing the one or more AGE products having similar wavelengths may be achieved by measuring their fluorescence lifetimes. In addition, the location of the one or more AGE products may be obtained by identifying their emission wavelengths.

Another aspect is a method of non-invasively pre-screening a patient for diabetes. The method comprises directing an excitation pulse at a region of the patient's skin to generate a fluorescence signal indicative of the composition of the patient's skin, measuring a transient intensity decay of the fluorescence signal, and comparing the measured transient intensity decay to a reference transient intensity decay value to diagnose the diabetic condition of the patient.

In some embodiments, the measured transient intensity decay is compared against a reference value according to the patient's age group.

In another embodiment, one or more AGE products are excited in the skin, the AGE products each generating a fluorescence signal having an identifiable wavelength and fluorescence lifetime. The method may further include measuring the fluorescence wavelength and lifetime, identifying a particular AGE product of interest via the fluorescence wavelength and lifetime, and comparing the AGE product of interest with a reference value for the AGE product of interest. The measured transient intensity decay may also be compared to a reference transient intensity decay value to diagnose the impaired glucose tolerance of the patient.

Furthermore, the excitation pulse may be controlled to vary wavelength, pulse width, repetition rate, peak and average power of the excitation pulse.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a time-resolved fluorescence optometric device in accordance with the present invention.

FIG. 2 is a graph of an exemplary excitation pulse over time.

FIG. 3 is a graph comparing the fluorescence magnitude of a healthy and diabetic patient over time.

FIG. 4 illustrates a diabetes pre-screening device and blood pressure monitor in accordance with the present invention.

FIG. 5 illustrates a clip of time-resolved fluorescence optometric device in accordance with the present invention.

FIG. 6 illustrates exemplary skin target locations for the device shown in FIG. 5.

FIG. 7 is graph of the energy rate emitted by an exemplary excitation pulse of light over time.

FIG. 8 is a schematic view of a Gaussian ultra short laser pulse incident on a simulated slab of tissue.

### DETAILED DESCRIPTION OF THE INVENTION

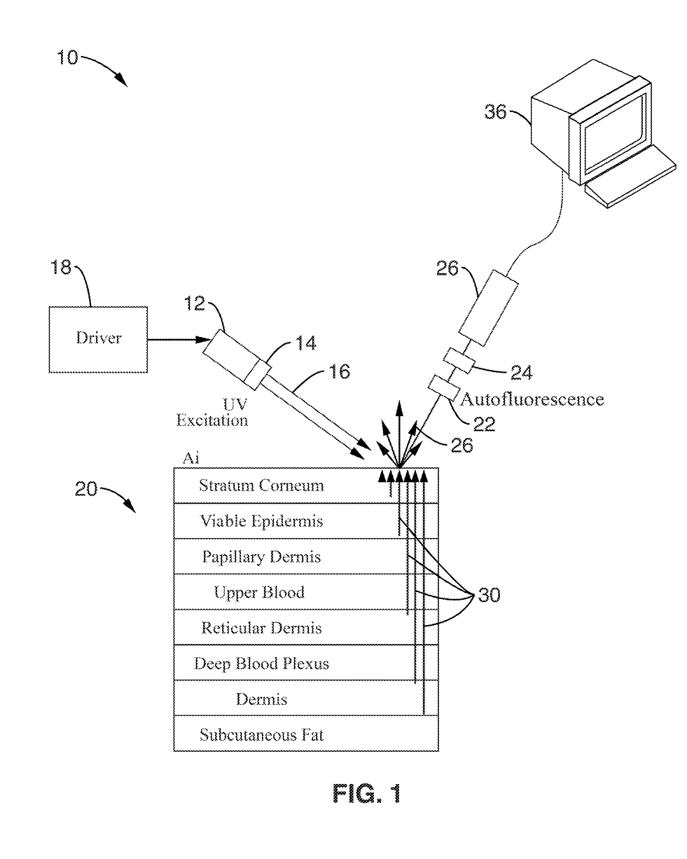

Referring more specifically to the drawings, for illustrative purposes the present invention is embodied in the apparatus generally shown in FIG. 1 through FIG. 8. It will be appreciated that the apparatus may vary as to configuration and as to details of the parts, and that the method may vary as to the specific steps and sequence, without departing from the basic concepts as disclosed herein.

Diabetes strongly affects the morphology, physiology, and autofluorescence characteristics of the human skin. For example, presence of diabetes mellitus is generally associated with measurably thickened skin among diabetic patients compared with their non-diabetic counterparts. Other characteristics include skin having a yellow hue, microangiopathy, and atrophic hyper pigmented macules on the shins, so-called diabetic dermopathy.

Therefore, light transport, and in particular transient light transport and time-resolved autofluorescence within the skin, differ from healthy subjects to diabetic patients. The change is significant enough to differentiate diabetic from healthy patients and to detect diabetes at an early stage and in a non-invasive manner using time-resolved skin autofluorescence.

In particular, some of the AGEs are fluorophores characterized by their (i) excitation and emission wavelengths, (ii) quantum yield and (iii) fluorescence lifetime(s). The fluorescence lifetime is the average time the electrons spend in their excited states. The quantum yield is the ratio of the number of photons emitted to the number absorbed. The fluorescence properties and locations of different endogenous fluorophores known to be present in human skin as well as those of AGEs can be found in the literature.

Table 1 summarizes the excitation-emission maxima of important biological chromophores. Collagen and elastin fluorescence is often determined using monochromatic excitation around 360 nm and emission in the spectral range from 415 nm to 440 nm.

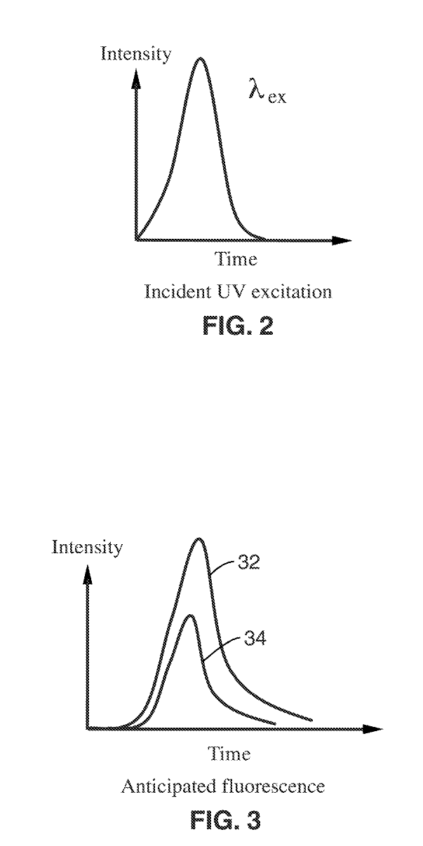

Referring now to FIG. 1, an optometric device 10 for non-invasively probing the inner structure of skin is schematically described in accordance with the present invention. The optometric device 10 comprises an excitation source 12 coupled to a first light guide 14, such as a fiber optic unit, to direct and transport excitation light pulses 16 to the skin 20 of a strategically selected area of the body. FIG. 2 illustrates the typical curve for incident excitation pulse intensity over time. Excitation source 12 is controlled by driver unit 18, and preferably comprises one or more pulsed sources of excitation electromagnetic (EM) waves, such as pulsed laser diodes or a pulsed light emitting diode (LED), a pulsed flash lamp, or similar device commonly used in the art. The fluorescent signal 26 is collected and transported by a second light guide 22 from the patient's skin 20 to a detector 28. It is appreciated that the excitation source 12 and detector 28 may be positioned to directly transmit and receive the signal to and from the patient's skin 20, thus the use of light guides 14, 22 are optional components of device 10, and may be removed to simplify the design.

The detector 28 may comprise a photomultiplier tube (PMT) using time correlated single photon counting, gated CCD spectrometer, streak cameras, single photon avalanche photo diode (SPAD) or similar device known in the art. In embodiments where the detector 28 comprises a PMT, a number of light guides 22 and PMT's can be positioned in an array to measure light at different positions and light paths through the patient's skin. Alternatively, a CCD spectrometer may be used without light guides 22, the CCD having an array of pixels that allows for imaging across a two dimensional area.

Since the reflected and fluorescent signals have different wavelengths, one or more optical filters or a device separating EM waves of different wavelengths 24, such as a monochromator, may be placed in line with the second light guide 22 and the detector 28 to separate the different signals. The detector 28 and driver unit 18 are synchronized by the processing unit 36.

The pulse of excitation light 16 is partially absorbed and scattered by the different skin layers 20. The absorbed light excites one or more fluorophores in the skin which in turn fluoresce 30. As shown in FIG. 3, the fluorescence curve 32 for a diabetic patient differs from the curve 34 for a healthy patient. For the same subject, the curve changes also with the patient's age and health. Abnormal changes will be indicative of a change in the subject's metabolism including but not limited to impaired glucose tolerance (IGT) or diabetes. The excitation pulses may be repeatedly applied to the skin at an arbitrary rate or frequency. The successive signal is preferably added, thus increasing the signal to noise ratio and the overall quality and reliability of the detected signals.

In one embodiment of the invention, the time-dependent reflected and fluorescence signals can be enhanced using index of refraction matching cream. This will limit the internal reflection within the skin. A photon that reaches the air-skin interface at an angle greater than the critical angle θ_{c}, defined by:
θ_{c}=arcsin(1/nₛₖᵢₙ)
where nₛₖᵢₙ is the refractive index of the skin, would be reflected back into the tissue. Typically, the critical angle for the air-skin interface is 41.8°. The angle of incidence of the excitation source 12 and detector 28 may also be varied to obtain optimal optical properties.

In another embodiment of the invention, the angle of incidence of the excitation (i.e. the angular orientation of the excitation source 12) could be varied during the course of the measuring procedure to measure the time-resolved bidirectional fluorescence, reflectance, and/or transmittance.

Similarly, the detector 28 orientation may be varied for collecting the fluorescence and reflectance signals at different angles. Alternatively, several liquid guides or fiber optics transporting the excitation pulse or the directional fluorescence, reflectance, and or transmittance signals could be installed at fixed angles.

The received energies from the detector 28 are then processed by the processing unit and computer software 36. The processing unit may comprise a computer, as shown in FIG. 1, or a small hand-held, portable device. In a preferred embodiment, the modified method of characteristics may be used in an algorithm to process the incoming signal from the detector, as described in further detail below. Because different fluorophores have different lifetimes, the time resolved approach of the present invention is capable of discriminating among fluorophores (that otherwise could not be distinguished using steady-state measurements).

The isolation of the individual fluorophores is preferably achieved through deconvolution of the transient signal, a process described in more detail in (O'Connor, D. V. and D. Phillips, 1984. Time-correlated Single Photon Counting. Academic Press, London) herein incorporated by reference in its entirety. The data may be processed using commercial software such as FluofitTM by PicoQuant GmBH to recover the skin fluorophores' lifetimes and their proportional contribution to the overall fluorescence signal from the skin. Fluorescence data may be compared and correlated with the currently available clinical laboratory values, including: subject age, glucose level, fasting blood glucose, HgA1C, and fructosamine for pre-screening and diagnosis of diabetes.

Additional information on the fluorophores locations, local concentrations, and skin morphology can be retrieved by processing the temporal signal directly provided by the detector using standard inverse techniques. The inversion consists of determining iteratively the radiation characteristics that minimize some difference between the measured and the calculated fluorescence, transmittance and/or reflectance. The calculation are performed using an algorithm, such as that for the modified method of characteristics, to solve the governing equation of electromagnetic wave transport through absorbing, scattering, and fluorescing media.

The number of excitation source elements 12 and the transmitted excitation light wavelength may be varied to alter the sensitivity of the device 10 including the analysis software. Several excitation laser diodes, light emitting diodes (LEDs), or pulsed flash lamps may be used to generate a pulse of excitation light having various wavelengths, pulse widths, repetition rate, and peak and average powers. For example, the pulse width is selected such that it is smaller than the fluorescence lifetime of the molecules or protein of interest. Since most fluorophores have more than a nanosecond lifetime, the ultra-short pulses will ideally have lengths less than a nanosecond. The frequency of the pulses may be at any rate, but is ideally at least 1 MHz, and by be as fast as the technology permits (e.g. 40 MHz) without imposing undue cost. Generally the faster the pulse rate, the lower the peak power. Thus, a range of 2.5 MHz to 40 MHz has been found to be optimal give the current state of technology. Similarly, for the frequency modulation technique, several modulation frequencies, peak and average power can be used. The excitation light may be UV, IR, visible light, or other form of electromagnetic wave commonly used in the art.

Time resolved fluorescence techniques include, but are not limited to, Time-Correlated Single Photon Counting (TCSPC), frequency modulation, gated photon counter, or the like.

In a preferred embodiment, UV light having a 370 nm excitation wavelength is used, as previous in-vitro studies have demonstrated that for most AGEs and digestible collagen cross-linked in particular, the excitation maximum varies between 335 nm and 370 nm and the emission maximum between 385 nm and 440 nm. An excitation wavelength of particular interest, in addition to the 370 nm currently used, is 335 nm corresponding uniquely to the AGE pentosidine. Other excitation and emission wavelengths can be used to avoid exciting or detecting fluorophores that may interfere with the fluorophores characteristics of the disease.

The intensity of the excitation light may also be varied to adjust sensitivity. As the intensity increases, the signal to noise ratio increases. However the light intensity it is limited by safety criteria. For this effect, excitation source 12 deposits very little energy but can carry enough power (average power of a few microwatts) for accurate detection.

One example of a preferred excitation source 12 is the PicoQuant diode Model PLS 370 is a class 1 laser product (LED), which requires no operator training, or any special equipment, such as eye protection, to operate the device. It is also safe to expose the human body to the non-ionizing radiation from this device. Moreover, the peak power of the device is 2.5 mW and average power of 5 μW at a 2.5 MHz repetition rate. The surface area of skin exposed to the excitation source is 2 cm in diameter or approximately 3.14 cm². In contrast, the solar irradiation deposited into the skin in the UV region from 370 nm to 390 nm measured at sea level, with the Sun at its zenith when the Earth is at an average distance from the Sun, is conservatively estimated at 6.76 W/m². Therefore, the excitation source at the peak power of 2.5 mW for 60 seconds at a wavelength of 380 nm corresponds to an exposure time of 71 seconds to sunlight. Consequently, the excitation source presents minimal risk as the probability and magnitude of harm or discomfort anticipated in the diagnostic measurement are not greater, in and of themselves, than those ordinarily encountered in daily life or during the performance of routine physical or psychological examinations or tests.

The optometric device 10 is preferably configured to be used on the patient's forearms, feet, earlobes, and hands. However, it may be used on any region on the patient's body that is readily accessible and appropriate light absorption characteristics.

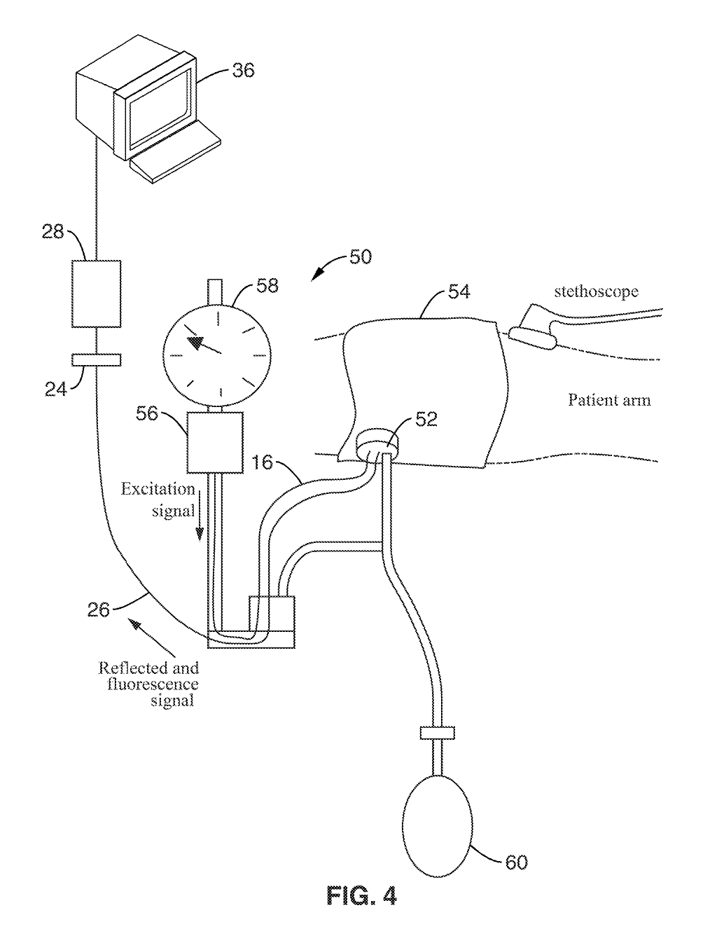

FIG. 4 illustrates an optometric device 50 integrated with a blood pressure monitoring system, wherein a system of fiber optic heads or light guides connected to one or more light sources and detector(s) will be placed at different locations on the forearm. This configuration has the added advantage that blood circulation is reduced in the forearm, thus limiting the absorption of the excitation light by blood. In addition, the numerous patients that have their blood pressure checked at each physician visit could have their fluorescence signal taken simultaneously. This would allow for universal screening, early detection and reduced complications of diabetes.

The optometric device 50 has a light guide 52 coupled to sphygmomanometer cuff 54 to be placed on the patient's arm. An excitation source 56 comprising a driver and one or more excitation elements (e.g. LEDS, laser diodes, or the like) may be coupled to a manometer 58 commonly used in blood pressure monitoring devices. While pressure is applied to the patient's arm via the sphygmomanometer cuff 54 and inflation bulb 60, an excitation signal 16 from the excitation source 56 is sent to the light guide unit 52. Alternatively, the excitation source may be directly incident on the patient's skin. The reflected and fluorescence signal 26 is then received by the detector for processing by computer 36.

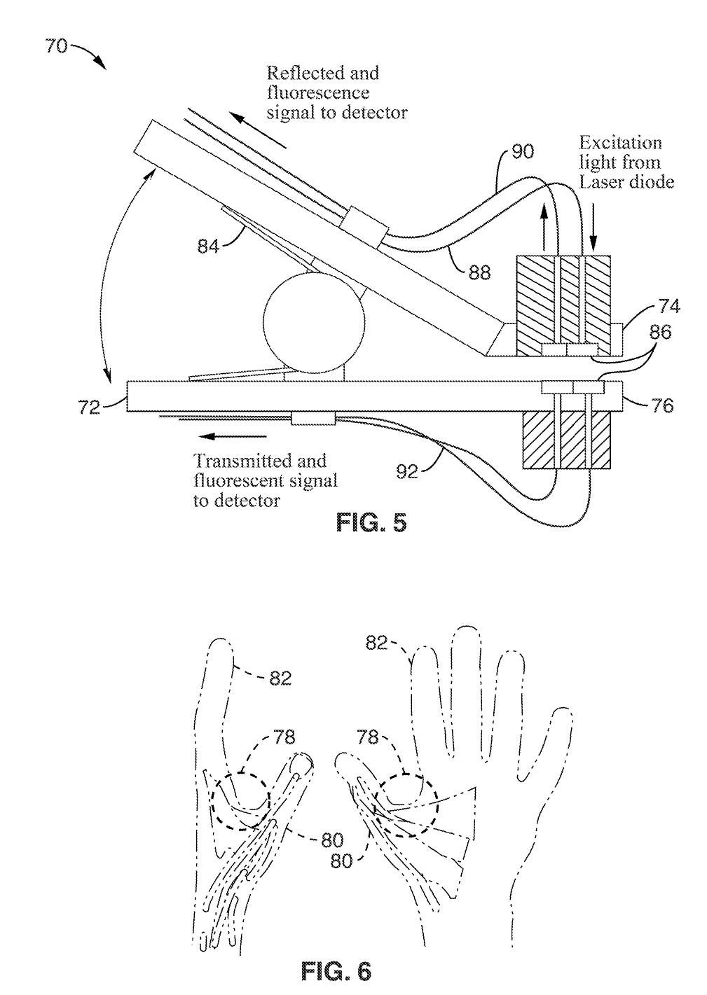

FIG. 5 illustrates another alternative embodiment comprising a clip-on optometric device 70. The clip-on optometric device 70 is configured to be positioned on opposing sides of the skin flap 78 between the thumb 80 and forefinger 82, as shown in FIG. 6. Alternatively, the clip on device 70 may be used on the patient's earlobes. In this region, blood vessels and fat are fairly limited and only skin is present. It also offers larger surface area for adequate optical contact between the non-invasive device 70 and the skin 80. Other possible sensing areas include the tongue and lips of the patient.

Moreover, the skin flap 78 and all of the above-mentioned sensing locations offer alternative tactics by enabling simultaneous time-resolved autofluorescence, reflectance, and transmittance measurements from both faces of the skin flap 78. As seen in FIG. 5, the device 70 comprises two opposing optical sensor heads: upper head 74 and lower head 76. The upper and lower heads 74, 76 are configured to be positioned on opposing sides of skin flap 78, and pressure may be applied to the skin flap 78 via spring 84 to ensure proper optical contact and tightness to outside light.

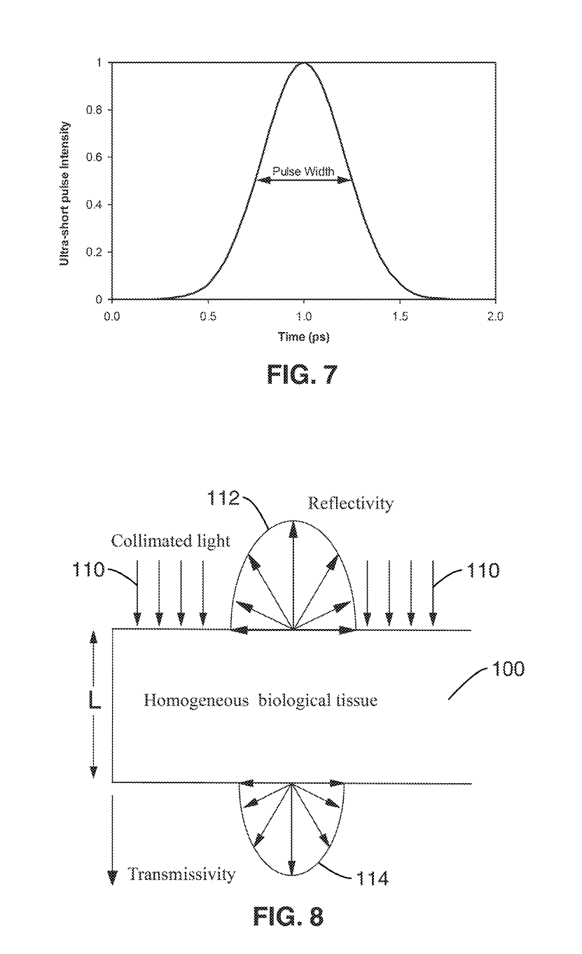

Each sensor head may have one or more light guides 86 for directing and transmitting optical signals. For example, upper head 74 may have fiber optics or light guides for directing excitation light 88, and for transporting the reflected and fluorescence signal 90 to the detector. The fluorescence, reflected, and transmittance signals are shown with reference to FIG. 8, which illustrates a one-dimensional thick slab 100 of biological tissues subjected to an incident collimated Gaussian ultra-short laser pulse 110 shown in FIG. 7. Correspondingly, the lower head 76 may have a light guide for directing the transmitted and fluorescence signal to the detector.

The additional measurements afforded by the optometric device 70 enable retrieval of the morphological properties of the skin thickness and optical properties of each layer, which are also affected by diabetes as previously discussed. Finally, the device 70 is easy to operate by a nurse and painless for the patient while assuring good optical contact between the probe and the skin.

The time-resolved fluorescence, reflectance, and transmittance data received from each patient may be collected and stored in a confidential database. This data may not only be used to validate the optical model and the simulations performed, but also develop a baseline of fluorescent signal for healthy patients. In addition, for each individual, the evolution of the fluorescence signal as a function of time may be recorded at each physician visit. Deviation from the healthy patient baseline would indicate abnormal metabolic changes affecting the skin optical and fluorescence properties and the occurrence or risk of diabetes mellitus. This would allow for universal screening, early detection and reduced complications.

Statistical, error management modeling, and signal processing methods commonly used in the art may also be used to process the data. The fluorescence signal is deconvoluted in order to isolate the contribution of individual fluorophores to the apparent cumulative signal. The overall performance of the system is assessed by measuring the sensitivity of the device as a function of false negative rate.

Generally, patients with longstanding diabetes will have a different fluorescence signal than age-matched controls. The differences appear in the values of the fluorescence lifetimes, individual fluorophores' contribution to the overall signal, their retrieved local concentrations, and/or fluorescence intensity in individuals who have had diabetes for longer periods of time and who are not in good control as evidenced by their clinical laboratory data (FGP, OGT and HgbA1C). Little to no overlap in the fluorescence values between affected individuals and age-matched controls is expected

The methods of the present invention may be used for pre-symptomatic testing, by identifying changes increase in the measured fluorescence compared to age-matched controls in patients developing diabetes. Alternatively, the methods of the present invention may provide insight into the causes of diabetes complications and may help assess the effectiveness of therapy of these complications.

The time-resolved fluorescence measurements of the present invention also enable identification of the fluorophores and measurement of their location and concentration in the skin, wherein the key fluorophores correlating with diabetes are distinguished to facilitate medical diagnostics.

A time-resolved fluorescence skin model may also be created that accounts for the absorption and fluorescence of protein in the skin (e.g., collagen, elastin), including AGEs accumulated in the skin to analyze the time-resolved fluorescence spectra. A reliable skin model may be developed by combining (i) the numerical tool described above for transport of light in multilayered turbid media, and (ii) optical and fluorescent characteristics of skin and its constituents reported in the literature across the UV and visible spectrum.

The optical skin model ideally accounts for (1) absorption by endogenous chromophores at the excitation and emission wavelengths which depend on skin complexion and patient's age, (2) autofluorescence by natural skin constituents, and (3) absorption and emission by accumulated fluorescent AGEs and other fluorophores. Time-resolved fluorescence characteristics include (i) lifetime, (ii) quantum yield, and (iii) excitation and emission wavelengths. The lifetimes and quantum yield of some fluorophores, such as pentosidine, HbA1c, and Hb-AGE, which remain unknown, may also be measured. First, small quantities may be isolated in order to characterize them using fluorescence lifetime spectrometers.

Finally, the optical model may be validated against experimental data collected from individual patients. As previously mentioned, the fluorescence characteristics of fluorophores, and in particular of bio-markers for diabetes such as pentosidine, HbA1c, and Hb-AGE, can be used for developing a reliable simulation tool in support of the medical diagnostics. The gradation of skin fluorescence as it correlates to the degree of glycemic control may be used to differentiate diabetic from healthy patients and therefore non-invasively detect diabetes at an early stage.

In an alternative embodiment, an optical model may be used accounting for more complex skin morphology. Instead of treating the skin as a series of plane parallel layers, the exact skin morphology will be obtained using image analysis software and a microphotograph of a cross-section of human skin. The Monte Carlo method may also be used instead of the modified method of characteristics, as it can simulate complex geometries and configurations and capture real physical conditions.

The method of the present invention has the following advantages: (1) non-invasive, (2) low cost, (3) allows for the motion of the subject thus making possible the study of infant, children, elderly, and patient with severe movement disorder, (4) uses non-ionizing radiation and therefore has no limits on the number of scans or pulses, (5) does not require fasting, (6) enables the determination of the location and concentration of fluorophores in the skin due to time-resolution. These pieces of information combined with lifetime measurement enable (7) the ability to distinguish between fluorophores. In addition, measurements are (8) not affected by skin conditions (tan, hair, or pigmentation) as much as steady-state fluorescence measurements, and (9) the device is easy to operate in clinical settings allowing for measurements to be done routinely by health professionals such as nurses at all physician visits or at least annually.

The proposed device offers a major breakthrough in the early detection of diabetes. It will provide a fast, safe, and non-invasive method to screen individuals for diabetes so that they can be diagnosed earlier leading to a decrease in complications and financial burden of this disease. In addition, this technology is portable, adapted to clinical settings, and can provide insight into the cause and efficacy of treatment of diabetic complications.

The potential benefit of this proposed research is to have a fast, non-invasive method to detect diabetes as well as assessing the degree of metabolic control of diabetes and follow the efficacy of therapy. This would greatly improve the state of the art of diagnosing diabetes as is does not require fasting or phlebotomy. In addition, this proposed device can be used to screen at risk individuals earlier therefore detecting diabetes early and avoiding complications. Finally, the device and the associated software could determine the nature and concentration of the skin fluorophores currently measured by performing an invasive skin biopsy

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
Optical properties of the seven layer skin model (Zeng et al., 1997)

| | | λ = 442 nm (excitation) | | | | λ = 520 nm (fluorescence) | | | |
|---|---|---|---|---|---|---|---|---|---|
| | thickness | | σₛ | κ | | | σₛ | κ | |
| Layer | μm | n | (cm⁻¹) | (cm⁻¹) | g | n | (cm⁻¹) | (cm⁻¹) | g |
| Air | | 1.0 | | | | 1.0 | | | |
| Stratum corneum | 10 | 1.45 | 190 | 2300 | 0.9 | 1.45 | 40 | 570 | 0.77 |
| Epidermis | 80 | 1.4 | 56 | 570 | 0.75 | 1.4 | 40 | 570 | 0.77 |
| Papillary dermis | 100 | 1.4 | 6.7 | 700 | 0.75 | 1.4 | 5 | 500 | 0.77 |
| Upper blood plexus | 80 | 1.39 | 67 | 680 | 0.77 | 1.39 | 24.5 | 500 | 0.79 |
| Reticular dermis | 1500 | 1.4 | 6.7 | 700 | 0.75 | 1.4 | 5 | 500 | 0.77 |
| Deep blood plexus | 70 | 1.34 | 541 | 520 | 0.96 | 1.34 | 181 | 500 | 0.96 |
| dermis | 160 | 1.4 | 6.7 | 700 | 0.75 | 1.4 | 5 | 500 | 0.77 |
| Subcutaneous fat | | 1.46 | | | | 1.46 | | | |

What is claimed is:

1. A method for non-invasively detecting diabetes in a patient; comprising:
directing a pulse of excitation light at a region of the patient's skin;
exciting one or more AGE products in the skin;
wherein excitation of said one or more AGE products generates a fluorescence signal;
detecting the fluorescence signal generated by the one or more AGE products; and
measuring the fluorescence signal as a function of time.

2. A method as recited in claim 1, wherein directing a pulse of excitation light comprises repeatedly directing a plurality of excitation pulses in succession at the region of the patient's skin.

3. A method as recited in claim 2, wherein the excitation pulses are subjected on the patient's skin at a rate of at least 1 MHz.

4. A method as recited in claim 3, wherein the successive pulses are added to increase the signal-to noise ratio of the signal.

5. A method as recited in claim 1, further comprising measuring the reflectance of the excitation pulse of light at the sensing region.

6. A method as recited in claim 5, further comprising measuring the transmittance of the excitation pulse.

7. A method as recited in claim 6, wherein the transmittance, reflectance, and time-resolved fluorescence measurements are performed simultaneously.

8. A method as recited in claim 1, further comprising:
storing measured fluorescence signal values acquired from a plurality of reference patients in a database.

9. A method as recited in claim 8, further comprising:
comparing the measured fluorescence signal values to key fluorophore values indicative of diabetes.

10. A method as recited in claim 9, wherein the compared fluorescence signal is used to assess the long term glycemic control in the patient.

11. A method as recited in claim 9, wherein the compared fluorescence signal is used to assess the impaired glucose tolerance in the patient.

12. A method as recited in claim 1, further comprising identifying one or more fluorophores from the measured in-vivo fluorescence signal.

13. A method as recited in claim 12, further comprising locating one or more fluorophores within the region of skin.

14. A method as recited in claim 12, wherein the fluorescence signal is deconvoluted to isolate the contribution of individual fluorophores to a cumulative signal.

15. An apparatus for detecting diabetes in a patient; comprising:
an excitation source configured to direct electromagnetic excitation energy at a region of the patient's skin;
a detector directed at the region of skin;
the detector configured to receive a fluorescence signal resulting from the excitation energy at the patient's skin; and
a processor configured to measure intensity decay of the fluorescence signal as a function of time to diagnose the diabetic condition of the patient.

16. An apparatus as recited in claim 15, wherein the excitation source comprises one or more LEDs.

17. An apparatus as recited in claim 16, further comprising one or more light guides for directing the excitation energy at the region of the patient's skin.

18. An apparatus as recited in claim 17, further comprising one or more light guides for directing the fluorescence signal emanating from the region to the detector.

19. An apparatus as recited in claim 15, wherein the excitation source is configured to repeatedly direct a plurality of excitation pulses in succession at the region of the patient's skin.

20. An apparatus as recited in claim 19, wherein the processor is further configured to measure the time resolved transmittance of the excitation pulses at the patient's skin.

21. An apparatus as recited in claim 20, wherein the processor is further configured to measure the reflectance of the excitation pulse at the patient's skin.

22. An apparatus as recited in claim 18, wherein the one or more light guides for directing the excitation energy are configured to be positioned on an opposing side of the region of skin opposite said one or more light guides for directing the fluorescence signal.

23. An apparatus as recited in claim 22, wherein the processor is further configured to perform transmittance, reflectance, and time-resolved fluorescence measurements simultaneously.

24. An apparatus as recited in claim 15, further comprising one or more optical filters displaced in the field of view of the detector.

25. An apparatus as recited in claim 15, wherein the excitation source is coupled with a sphygmomanometer cuff of a blood pressure monitoring device such that excitation energy may be directed while pressure is being applied to the region of the patient's skin.

26. A method for performing time-resolved fluorescence measurements to diagnose the diabetic condition of a patient; comprising:
directing an excitation pulse at a region of the patient's skin;
exciting a portion of the patient's skin as a result of the excitation pulse at the region to generate a fluorescence signal indicative of the composition of the patient's skin;
detecting the fluorescence signal generated by the excitation pulse; and
measuring a transient intensity decay of the fluorescence signal to determine the diabetic condition of the patient.

27. A method as recited in claim 26, wherein exciting a portion of the patient's skin comprises exciting one or more AGE products in the skin;
the one or more AGE products each generating a fluorescence signal.

28. A method as recited in claim 27, wherein directing an excitation pulse comprises repeatedly directing a plurality of ultra short pulses in succession at the region of the patient's skin.

29. A method as recited in claim 27, wherein directing an excitation pulse comprises repeatedly directing a frequency modulated light at the region of the patient's skin.

30. A method as recited in claim 28, wherein signals from the successive pulses are added to increase the signal-to noise ratio of the signal.

31. A method as recited in claim 28, further comprising measuring the reflectance of the excitation pulse.

32. A method as recited in claim 28, further comprising:
distinguishing between the one or more AGE products by measuring their emission wavelengths.

33. A method as recited in claim 32, further comprising:
distinguishing the one or more AGE products having similar wavelengths by measuring their fluorescence lifetimes.

34. A method as recited in claim 28, further comprising:
identifying the location of the one or more AGE products by identifying their emission wavelengths.

35. A method as recited in claim 28, wherein the fluorescence signal is deconvoluted to isolate the contribution of individual fluorophores to a cumulative signal.

36. A method as recited in claim 31, further comprising measuring the transmittance of the excitation pulse.

37. A method as recited in claim 26, further comprising:
storing measured intensity decay values acquired from a plurality of reference patients in a database.

38. A method as recited in claim 37, further comprising:
comparing the measured intensity decay to key fluorophore values corresponding to diabetes.

39. A method as recited in claim 38, wherein the compared intensity decay is used to assess the long term glycemic control in the patient.

40. A method as recited in claim 38, wherein the compared intensity decay is used to assess the patient's risk of developing diabetes.

41. A method of non-invasively pre-screening a patient for diabetes, comprising:
directing an excitation pulse at a region of the patient's skin to generate a fluorescence signal indicative of the composition of the patient's skin;
measuring a transient intensity decay of the fluorescence signal; and
comparing the measured transient intensity decay to a reference transient intensity decay value to diagnose the diabetic condition of the patient.

42. A method as recited in claim 41, wherein the measured transient intensity decay is compared against a reference value according to the patient's age group.

43. A method as recited in claim 42, wherein directing an excitation pulse comprises exciting one or more AGE products in the skin;
the one or more AGE products each generating a fluorescence signal having an identifiable wavelength and fluorescence lifetime.

44. A method as recited in claim 43, further comprising:
measuring the fluorescence wavelength and lifetime;
wherein comparing the measured transient intensity decay comprises identifying a particular AGE product of interest via the fluorescence wavelength and lifetime; and
comparing the AGE product of interest with a reference value for the AGE product of interest.

45. A method as recited in claim 41, further comprising:
controlling the excitation pulse to vary wavelength, pulse width, repetition rate, peak and average power of the excitation pulse.

46. A method as recited in claim 41, wherein the measured transient intensity decay is compared to a reference transient intensity decay value to diagnose the impaired glucose tolerance of the patient.
